SEQUENCE LISTING (12 sequences)

SEQ ID NO: 1 - DNA, 21 bp, Artificial Sequence
  HNRNPA1 gene 83bp Amplicon Forward Primer
gggctttgcctttgtaacctt

SEQ ID NO: 2 - DNA, 23 bp, Artificial Sequence
  HNRNPA1 gene 83bp Amplicon Probe
tgacgaccatgactccgtggata

SEQ ID NO: 3 - DNA, 22 bp, Artificial Sequence
  HNRNPA1 gene 83bp Amplicon Reverse Primer
tgtggccattcacagtatggta

SEQ ID NO: 4 - DNA, 20 bp, Artificial Sequence
  HNRNPA1 gene 231bp Amplicon Forward Primer
ggacccatgaagggaggaaa

SEQ ID NO: 5 - DNA, 22 bp, Artificial Sequence
  HNRNPA1 gene 231bp Amplicon Probe
tttggaggcagaagctctggcc

SEQ ID NO: 6 - DNA, 21 bp, Artificial Sequence
  HNRNPA1 gene 231bp Amplicon Reverse Primer
gcttggctgagttcacaaatc

SEQ ID NO: 7 - DNA, 21 bp, Artificial Sequence
  Chromosome 2p15 87bp Amplicon Forward Primer
cagcgttggtagatcctgaca

SEQ ID NO: 8 - DNA, 24 bp, Artificial Sequence
  Chromosome 2p15 87bp Amplicon Probe
tctggccacacttgagttccatgg

SEQ ID NO: 9 - DNA, 19 bp, Artificial Sequence
  Chromosome 2p15 87bp Amplicon Reverse Primer
ctgcgagtgctgcgagaag

SEQ ID NO: 10 - DNA, 21 bp, Artificial Sequence
  Chromosome 2p15 250bp Amplicon Forward Primer
ttccgacagacctttccactc

SEQ ID NO: 11 - DNA, 20 bp, Artificial Sequence
  Chromosome 2p15 250bp Amplicon Probe
ccaccgtctgtggcctgagg

SEQ ID NO: 12 - DNA, 19 bp, Artificial Sequence
  Chromosome 2p15 250bp Amplicon Reverse Primer
aggtgaggcgcgtaaagga

(19) United States
(12) Patent Application Publication
(10) Pub. No.: US 2013/0078639 A1
(43) Pub. Date: Mar. 28, 2013

(54) SYSTEM AND METHOD OF DETECTING AND CORRECTING FOR NUCLEIC ACID DAMAGE

(71) Applicant: International Genomics Consortium, Phoenix, AZ (US)

(72) Inventor: Scott Morris, Phoenix, AZ (US)

(73) Assignee: INTERNATIONAL GENOMICS CONSORTIUM, Phoenix, AZ (US)

(21) Appl. No.: 13/625,791

(22) Filed: Sep. 24, 2012

Related U.S. Application Data
(60) Provisional application No. 61/538,426, filed on Sep. 23, 2011.

(51) Int. Cl. C12Q 1/68; G06F 19/18

(52) U.S. Cl. CPC C12Q 1/686; G06F 19/18
USPC 435/6.12; 702/21

(57) ABSTRACT

The present disclosure describes a method to estimate a geometric parameter to describe the degradation pattern (i.e. the proportion of bases that are damaged) in a sample. Using the values provided by the described systems and methods, researchers can estimate the proportion of undamaged fragments that are a certain base pairs in length or can estimate the number of errors within a fragment of certain base pairs in length.

### CROSS-REFERENCE TO RELATED APPLICATIONS

This application claims priority to U.S. Provisional Patent Application 61/538,426 filed on Sep. 23, 2011 and entitled SYSTEM AND METHOD FOR ASSESSING NUCLEIC ACID QUALITY, the entirety of which is hereby incorporated by reference herein.

### SEQUENCE LISTING

The present application is being filed along with a sequence listing in Electronic format. The Sequence Listing is provided as a file entitled IGNCO-003A.TXT, created Sep. 21, 2012, which is approximately 3 kb in size. The information in the electronic format of the sequence listing is incorporated herein by reference in its entirety.

### FIELD OF THE INVENTION

The disclosure relates to systems and methods for assessing preserved nucleic acid samples, and methods for correcting against any biases which nucleic acid degradation may introduce into nucleic acid assessment. More particularly, the disclosure relates to systems and methods for determining the amount of nucleic acid degradation in cells from preserved tissue samples.

### BACKGROUND OF THE INVENTION

Surgery provides health care professionals a unique opportunity to obtain samples from a patient. However, as surgery is time-critical, many samples obtained during surgery must be preserved for later evaluation rather that evaluated immediately. Additionally, many surgical samples are archived for later analysis and reference.

The most commonly used method of preservation of tissues for later analysis or archiving is formalin fixed, paraffin embedding (FFPE). Often, only FFPE samples are available from procedures involving routine treatment of cancers and other diseases.

Because of the ease of use, the availability of the reagents and the durability of preserved samples, large archives of FFPE samples exist. These archives represent a valuable source of sample material for research (Wen-Yi Huang, Timothy M. Sheehy, Lee E. Moore, Ann W. Hsing and Mark P. Purdue. Cancer Epidemiol Biomarkers Prev April 2010 19: 973)

The processes of creating FFPE samples, and of extracting DNA or RNA from said samples, can cause substantial damage to sample nucleic acids. This damage can involve double-strand breaks, abasic sites, intrastrand cross-linking and interstrand crosslinking. In studies that examine nucleic acid regions of various sizes, these processes introduce bias against larger fragments (i.e. fewer larger fragments are detectable than smaller ones).

There are few readily accessible methods available to the public for precisely determining the extent of damage to a nucleic acid sample, and even fewer methods provide the required precision to compare many samples taken from different hospitals and with different surgeons. Electrophoresis and bioanalyzers, for example, may only detect strand breaks. The quantitative polymerase chain reaction (qPCR) ACq method is used for some quantification, but is on its own very sensitive to reaction efficiency, does not always produce biologically relevant results, and the results it does produce may not have a linear relationship to nucleic acid sample quality.

### SUMMARY OF THE INVENTION

The present disclosure provides methods of assessing the integrity of nucleic acids such as DNA and RNA derived from formalin fixed, paraffin embedded (FFPE) samples.

In some embodiments this assessment comprises measuring total nucleic acids (i.e. by fluorescence), then measuring one or more targets (i.e. by quantitative PCR or ‘qPCR’). In some embodiments this assessment comprises measuring two or more fragments of various sizes (i.e. by qPCR). For example, a fragment about 70 bp and one around 250 bp may be used.

In some embodiments, the measurements are used to determine which among a set of FFPE samples are suitable for analysis. In some embodiments, the measurements are used to determine how much nucleic acid of a given sample should be used in an analysis reaction, such as a next-generation sequencing reaction. In some embodiments, the measurements are used to evaluate or to eliminate biases in analysis that result from damage to FFPE preserved nucleic acids.

In some embodiments a method of performing manipulations on a sample of interest is taught. The method may comprise providing nucleic acids from a sample of interest. The amount of total nucleic acids provided from the sample may be measured. A preselected region of the nucleic acids may be amplified to form amplicons having a predetermined length such that only nucleic acids that do not contain damage are amplified. The amount of amplicons generated may be measured. The amount of amplicons obtained by amplification of said nucleic acids may be compared to a standard curve reflective of amounts of undamaged template that would yield similar amounts of template. An amount of undamaged nucleic acids that would yield the measured amount of amplicons generated is determined. The total nucleic acid amount may be compared to the determined equivalent amount of undamaged nucleic acids corresponding to the amplification level of the nucleic acids from the sample. The comparison may be indicative of a proportion of base positions in the nucleic acids from a sample of interest template for the amplicons that are damaged. A further manipulation may be performed on said sample of interest if the proportion is below a threshold value.

In some embodiments a method of performing manipulations on a sample of interest is taught. The method may comprise providing nucleic acids from a sample of interest. The amount of total nucleic acids provided from the sample may be measured. A preselected region of the nucleic acids may be amplified to form amplicons having a predetermined length such that only nucleic acids that do not contain damage are amplified. The amount of amplicons generated may be measured. The amount of amplicons obtained by amplification of said nucleic acids may be compared to a standard curve reflective of amounts of undamaged template that would yield similar amounts of template. An amount of undamaged nucleic acids that would yield the measured amount of amplicons generated is determined. The total nucleic acid amount may be compared to the determined equivalent amount of undamaged nucleic acids corresponding to the amplification level of the nucleic acids from the sample. The comparison may be indicative of a proportion of base positions in the nucleic acids from a sample of interest template for the amplicons that are damaged. The extent of damage to the tissue sample may be determined by comparing the assessed proportion to a threshold value.

In some embodiments a method of performing manipulations on a sample of interest is taught. The method may comprise providing nucleic acids from a sample of interest, amplifying a preselected region of the nucleic acids to form a first amplicon of a predetermined length n1, and amplifying a preselected region of the nucleic acids to form a second amplicon of a predetermined length n2. The method may comprise measuring the amount of amplified nucleic acids from of said the first and the second amplicon. The method may comprise comparing the amount of the first and the second amplicon generated, wherein the comparison is indicative of a proportion of base positions in the template for the amplicons that are damaged. The method may comprise performing a further manipulation on said sample of interest. In some embodiments the manipulation may be performed if the proportion is below a threshold value.

In some embodiments a method of performing manipulations on a sample of interest is taught. The method may comprise providing nucleic acids from a sample of interest, amplifying a preselected region of the nucleic acids to form a first amplicon of a predetermined length n1, and amplifying a preselected region of the nucleic acids to form a second amplicon of a predetermined length n2. The method may comprise measuring the amount of amplified nucleic acids from of said the first and the second amplicon. The method may comprise comparing the amount of the first and the second amplicon generated, wherein the comparison is indicative of a proportion of base positions in the template for the amplicons that are damaged. The method may comprise determining the extent of damage to the tissue sample. In some embodiments the method comprises comparing the assessed proportion to a threshold value.

### BRIEF DESCRIPTION OF THE DRAWINGS

FIG. 1 is a graph illustrating a scatterplot of values of the proportion (p) of damaged RNA bases (p) determined using primers specific to β-actin (ACTB) and 18S RNA transcripts.

FIG. 2 is a point graph comparing the proportion (p) of damaged DNA bases to the proportion (p) of damaged RNA bases from 10 specimens.

FIG. 3 is an illustration of the use of a p value determination to correct amplicon count numbers.

FIG. 4 is a bar graph showing a boxplot of DNA and RNA yields from Formalin Fixed Paraffin Embedded (FFPE) tissue samples.

FIG. 5 is a point graph illustrating a scatterplot of DNA and RNA yields from FFPE samples.

FIG. 6 is a bar graph illustrating a boxplot of OD measurements of DNA and RNA.

FIG. 7 is an image of a gel showing a comparison of values of p with electrophoretic measurements of nucleic acid damage.

### DETAILED DESCRIPTION

Embodiments relate to systems and methods for assessing the quality of tissues that have been taken from a subject. This may involve both assessing the extent of any damage to the tissues as well as determining the quantity of intact nucleic acid present in the tissues. As described below, the present disclosure details methods related to the assessment of nucleic acid integrity and quantity within these tissues having undergone an undetermined amount of sample degradation.

In some embodiments, the systems and methods disclosed herein enable one to evaluate the degree of damage to nucleic acids extracted from an FFPE-preserved sample. In some embodiments the degree of damage to nucleic acids within the sample may be used as a proxy representation of the degree of damage to non-nucleic acid constituents of the sample, such as proteins. Thus, by measuring the amount of damage to the DNA or RNA in the sample, it is possible to approximate the amount of damage that has been done to the tissue, or proteins, within that tissue sample.

In some embodiments, nucleic acids, such as DNA or RNA, are extracted from an FFPE sample. Primers selected to amplify suitable regions of the nucleic acids isolated from the sample, discussed in more detail below, are applied to generate a first amplicon of length n. Without being bound by any particular theory, it is expected that amplicons will only be generated from undamaged template nucleic acids. Damaged templates, particularly fragmented templates, will not be capable of generating amplicons from the selected primers. In some embodiments, standard curves are generated to facilitate analysis. Undamaged template nucleic acids, at amounts which correspond to the amount of template which would be available in a sample having no damaged nucleic acids, may be used to form such a standard curve. In some embodiments, the amplification level of the amplicon in the nucleic acids extracted from the sample may be compared to the amplification level of the amplicon generated from the undamaged templates in the standard curve to get a measure of the effective undamaged template amount (x) in nucleic acids derived from a sample. In some embodiments, the total amount of nucleic acids derived from a sample (c) may be determined, for example by placing a sample in a spectrophotometer. In some embodiments, a second amplicon is generated having a length n₂ which is different form the length n of a first amplicon, and the effective undamaged template amount (x₂) is similarly determined for the second amplicon.

Using the methods and systems disclosed herein, one may compare the effective undamaged template amount (x) to the total amount of nucleic acids derived from a sample (c), factoring in the length (n) of the amplicon, to assess the chance (p) of a base in the nucleic acids derived from said sample being damaged.

Similarly, using the methods and systems disclosed herein, one may compare the effective undamaged template amount (x) to a second effective undamaged template amount (x₂) determined for the second amplicon, factoring in the difference in lengths of the two amplicons, to assess the chance (p) of a base in the nucleic acids derived from said sample being damaged.

Having determined the chance p for nucleic acids derived from a given sample, one may perform a number of manipulations on either the sample or the nucleic acids derived therefrom. In some embodiments, one may include or exclude a sample, or its nucleic acids, based upon the determination of the chance that nucleic acids derived from said sample are damaged. In some embodiments one may adjust the amount of nucleic acids added to a sample so that an effective amount of undamaged nucleic acids are added to a downstream application. In some embodiments the downstream application is nucleic acid sequencing.

Through the methods disclosed herein, one can correct for defects in previous nucleotide assessment methods. Through the implementation of the methods disclosed herein, one may create a p metric, that is a determination of the percentage of bases in a nucleic acid fragment that are damaged. Upon obtaining a p value, one may further assess the proportion of sites of a nucleic acid of a given length that are damage free. Similarly, one may further assess the number of damaging events on average that exist within a region of a given size.

The nucleic acid degradation to be evaluated may result from nucleic acid extraction from a sample, such as a sample obtained from an individual in surgery and preserved as an FFPE sample. However, the nucleic acid may be derived from any number of sources, such as animal sources, plant sources, fungal sources, bacterial sources, or other the nucleic acid may be derived from another organism. The nucleic acid may be of eukaryotic origin, eubacterial origin, archaeal origin, or viral origin. The nucleic acid may be artificially synthesized. The nucleic acid may be obtained from an individual directly, or may be obtained from an environmental sample, crime scene sample, archaeological sample, or any source which may comprise nucleic acids of unknown integrity.

The nucleic acid may be preserved in a sample, such as a flash-frozen sample or otherwise frozen sample. The sample may be chemically preserved, such as by treating the sample with a fixative such as a cross-linking agent, such as dimethyl suberimidate, the N-Hydroxysuccinimide-ester crosslinker BS3, formaldehyde, EDC, SMCC, Sulfo-SMCC. The sample may be fixed in formalin. The sample may be embedded in a solid matrix such as paraffin. In some embodiments, the sample is a preserved FFPE sample from which nucleic acids are obtained.

The nucleic acid may be isolated from a sample and stored. Isolated samples may be lyophilized, stored in a buffer, or stored in a solution largely comprising ethanol. The nucleic acids may be stored at room temperature, at 4° C. or frozen, for example in a freezer at −20° C. or −80° C., or stored in a vial in contact with liquid nitrogen.

The nucleic acid may be isolated from a sample any number of ways. Nucleic acid extraction may be tailored to the sample source, or may be performed using standard phenol-chloroform-ethanol, phenol-ethanol, or acid-base extraction method. In some embodiments a heptane deparaffinization option with increased volume is used. In some embodiments a miRNA extraction protocol is used. The methods disclosed herein are not limited to any particular nucleic acid extraction method. In some embodiments, both RNA and DNA are simultaneously extracted from FFPE samples as follows: an excess of heptane (for example, 1.4 mL) is added to 10 μm sections of the sample, and the sample is heated in order to dissolve the paraffin within and around the specimen. Methanol is added to precipitate nucleic acid, and the sample is centrifuged to pellet the precipitant and the pellet is washed with ethanol. A lysis buffer and proteinase K are added, and the sample is centrifuged causing DNA to form a pellet at the bottom of the tube while the RNA remains in the supernatant. The DNA is then extracted via the Allprep FFPE kit (Qiagen #80234), and the RNA is extracted by the Highpure miRNA kit (Roche #05080576001).

The nucleic acid sample may comprise nucleic acids damaged in a number of ways. Nucleic acids may, for example, suffer double strand breaks, single strand breaks, abasic sites, intra-strand cross-linking and inter-strand crosslinking, or base modification. The damage may result from the treatment of the sample in preparation for storage, during storage as a result of sample storage conditions, or the process of extraction. Damage may occur before the nucleic acid is isolated from a sample.

One embodiment is a method for obtaining a metric to assess the quality of DNA and RNA derived from formalin-fixed paraffin-embedded (“FFPE”) specimens. It can be assumed that formalin fixation causes damage that is geometrically distributed throughout the genome of cells within the fixed sample. The damage can occur in multiple forms, which may include double stranded breaks, abasic sites, intrastrand cross links and inter strand cross links. In order to assess the extent of the damage, a fragment of known size from the genome can be amplified via qPCR. Because qPCR is sensitive to damage, the number of copies detected can be reduced to only include those copies that are fully intact. This result can be compared to another method (i.e. spectrophotometry) that can detect all copies of the gene regardless of damage. This can be similar to the ACq method, but can allow compensation for reaction efficiency and can present the results in a biologically-relevant number that allows direct predictions of assay performance by other methods.

In some embodiments the FFPE specimens are three months to one year old. The specimen may be, for example, 0.3 cm to 1 cm on each dimension. In one embodiment, the samples may be collected at a plurality of hospitals, each with its own varying methods of sample collection. Alternatively, the samples may be from a single source with a uniform collection and preservation method. In some embodiments the specimens may be fully embedded and may not have had previous slides cut. In some embodiments no preserved specimen face has been exposed to air.

In some embodiments, the nucleic acid may be evaluated as follows. Total nucleic acid levels in a sample may be measured by any number of methods known in the art. For example, RNA may be measured using the RIBOGREEN® RNA Quantitation Kit (Molecular Probes, Eugene Oreg.), the Bioanalyzer (Agilent), some other fluorescent quantification method or UV spectrophotometry. DNA may be measured using PICOGREEN® RNA Quantitation Kit (Molecular Probes, Eugene Oreg.), the Bioanalyzer (Agilent), some other fluorescent quantification method or UV spectrophotometry. Nucleic acid levels may also be measured using any number of nonspecific means, such as spectrophotometric measurements or the use of a non-sequence specific nucleic acid intercalating agent such as Ethidium Bromide. In some embodiments the method measures total nucleic acid levels of double and single stranded nucleic acids in a sample. In some embodiments the method distinguishes DNA from RNA so that the level of one of either DNA or RNA can be measured specifically.

Specific, amplification-quality nucleotide template may be measured by, for example, performing qPCR to amplify a fragment from a nucleic acid sample. Using standard techniques one can determine the amount of quantifiable template in a given sample. For example, one may amplify a fragment of around 80 bp by qPCR, for example using a Qiagen® QuantiFast Probe Assay QF00531209 kit that amplifies an 85 bp fragment of human 13-actin. Alternatively, a Qiagen® QuantiFast Probe Assay QF00530467 kit may be used to amplify an 80 bp fragment of human 18S ribosomal RNA. In one embodiment, after such amplification, appropriate standards can be used to create a standard curve and fit the sample to the standard curve. Other amplicons or fragment sizes may be selected according to the method disclosed herein.

### Standard Curve Generation

A series of standards may be run to allow direct comparison of the amplification level of an amplicon generated from nucleic acids extracted from a sample, such as an FFPE sample, to the amplification level of an amplicon generated from a template of known amount and quality. These standards may be, for example, DNA known to be diploid, synthetic nucleic acid, DNA/RNA from cell lines, nucleic acid extracted from tissue, or an amount of template corresponding to the molar amount of template predicted to be present in a given nucleic acid type. The standards may be diluted to varying concentrations to encompass all concentrations of template or of undamaged template expected to be found in nucleic acids extracted from the specimens. In one embodiment, a diploid DNA or cell line standard may be run at concentrations of 100 ng/μL, 10 ng/μL, 1 ng/μL and 0.1 ng/μL. The standards may be subject to qPCR amplification of an amplicon or amplicons of interest. The Ct value of these qPCR amplifications may be recorded. These Ct values may be used to generate standard Ct values that correspond to known amounts of undamaged template. Linear regression of logarithm-transformed values may be used to create a standard curve to fit Ct values corresponding to amplification levels of nucleic acids from other samples of unknown concentration, such as those which have been run simultaneously. The concentration of the unknown samples may be determined, for example, by inserting their Ct values into the regression equation obtained from standards to calculate the effective concentration of undamaged template in the nucleic acids derived from a given unknown sample. Multiple replicates of each standard and nucleic acids from each unknown sample may be run. The Ct value may be collected at a value that is known to obtain consistent results, or the Ct value may be obtained by selecting the Ct value where variance within the regression of the standards is minimal. Other methods of determining when to record a Ct value are known in the art and may be used in embodiments of the systems and methods disclosed herein. Similarly, other methods of quantifying amplification amounts from PCR reactions may be used to evaluate amplificaiton levels, form standard curves and evaluate nucleic acids isolated from samples consistent with the systems and methods disclosed herein.

The observed amplification level of an amplicon can be correlated with the amplification level determined for a known standard, for example by using the method disclosed above. Upon completing this correlation, one may conclude that the amplifiable template of undamaged nucleic acids in the sample that gave rise to that amplicon is present at a level equal to that of the template in the standard.

In some embodiments, it is important that regions interrogated by primers are present at consistent levels across a wide variety of nucleic acids expected to be assayed. In the case of DNA, a region that does not undergo or is unlikely to undergo copy number gains/losses relative to the rest of the genome is preferred. In some embodiments, in order to determine a suitable region, one may obtain copy number results across the genome (e.g. by microarray) and determine which regions tend to have a copy number most similar to the median copy number of all regions. In some embodiments, one may be examining malignant specimens, and chromosome 2 may be selected as it rarely experiences copy number events across a wide range of cancers. In some embodiments primers are selected which direct the amplification of an amplicon of suitable size from a template consisting of DNA of human chromosome 2.

In the case of non-malignant diploid specimens, the region selected may be of little importance.

In some embodiments the nucleic acids derived from a sample are RNA. In some such embodiments, the region selected should be a gene that is known to be expressed at similar levels throughout the types of tissues expected to be assayed. One may conduct next-generation RNA sequencing (e.g. on the Illumina Hiseq) to understand the expression profiles of many genes, and may select the gene with the lowest variance in the RPKM value. HNRNPA1 may be selected due to its extremely low variance between specimens. Known reference genes may be used, such as ACTB or 18S. Once a gene is selected, primers may be designed to amplify a region near the center of the gene as this region is less sensitive to exonucleases.

In some embodiments, the total quantity of nucleic acid (e.g. in ng/μL) may be determined. In some embodiments the total quantity may be determined by a method that non-specifically measures RNA or DNA, such as a method involving PICOGREEN® or RIBOGREEN®, or spectrophotometry. The amount of amplifiable material may be determined by measuring the Ct value by qPCR and fitting it to linear regression to determine quantity (e.g. in ng/μL), for example using methods discussed above. Upon determining the total (c) and amplifiable (x) nucleic acid levels in a sample, one may calculate the proportion of base positions that are damaged (p) for a given amplicon length (n). In some embodiments such a determination may be accomplished using the disclosed equation 1:

$$p = 1 - \sqrt[n]{\frac{x}{c}} \qquad ( 1 )$$

In some embodiments, the nucleic acid may be evaluated as follows: Two fragments of different sizes in a nucleic acid sample, referred to as amplicon 1 and amplicon 2, may be amplified by qPCR. Appropriate standards are used and measured values are fit to the standard, for example, by regression. Regression may be carried out as previously described, with the regression for each fragment occurring separately. In the case were a multiplexed assay is used, such as Taqman®, a regression can be conducted for each component assay. Methods of generating standards in qPCR and of fitting sample qPCR results to standards are known to those of skill in the art.

By the disclosure of an embodiment of the method herein, the ratio of the quantification result of the two amplicons may be used as an indicator of nucleotide damage, wherein a larger ratio of the large amplicon to the small one indicates more intact nucleic acid. For example, one may use amplicons of about 70 bp and about 250 bp in length. However, it should be realized that amplicons of other lengths are also contemplated by this disclosure. For example, amplicons of 50, 60, 70, 80, 90, 100, 110, 120, 130, 140, 150, 160, 170, 180, 190, 200, 225, 250, 275, 300, 350, 400 or more than 400 base pairs in length may be used.

Similarly, in some embodiments, the nucleic acid may be evaluated as follows. The sizes n₁ and n₂ of the amplicons and the quantification levels x₁ and x₂ of amplicons 1 and 2 respectively are determined. In some embodiments such a determination of amplification levels is made using the methods described above. In some embodiments, commercially available reagents and methods disclosed herein may be used to determine such levels (e.g. by conducting a 2-plex Taqman® assay then fitting by regression). In some embodiments, one can according to the method disclosed herein determine the proportion of bases damaged (p), for example by using the disclosed equation 2:

$$p = 1 - \sqrt[{n}_{1} - {n}_{2}]{\frac{{x}_{1}}{{x}_{2}}} \qquad ( 2 )$$

Similarly, using this method one may estimate the number (t) of intact fragments greater than a length (m) by using the disclosed equation 3:

t=x₁(1−p)^{m-n}^{₂} (3)

In some embodiments, the methods disclosed herein may be used to determine which samples are suitable for a downstream analysis. A desired ratio or value of p may be selected as a threshold that defines what samples are acceptable for downstream analysis. Samples on the wrong side of such a threshold are excluded from further analysis. For example, a threshold of 0.25%, 0.5%, 0.75%, 1.0%, 1.25%, 1.50%, 1.75%, 2.0%, 2.5%, 3.0%, 4.0%, 5.0%, 10%, or more than 10% may be used such that samples obtaining values above the threshold are excluded from downstream analysis.

In some embodiments, the methods disclosed herein may be used to determine how much of a given sample should be added to a given analysis assay to provide a target amount of amplifiable or otherwise non-degraded nucleic acid for a downstream analysis.

While the present invention has been described in some detail for purposes of clarity and understanding, one skilled in the art will appreciate that various changes in form and detail can be made without departing from the true scope of the invention.

### Model Derivation

In some embodiments one obtains some measured concentration of total RNA or DNA, c, and some measure of intact nucleic acid template in nucleic acids derived from a sample, x, such that the length of the intact nucleic acid being measured is n nucleotides. This may be modeled as a series of Bernoulli trials such that there is a probability of p that a damaging event will occur in any given nucleotide base position along the nucleic acid polymer and probability of 1−p that there will not be a damaging event. Thus, one may calculate the probability that no damaging events occur over a region of n nt long as:

P(no damage)=(1−p)ⁿ

wherein Z is defined as the proportion of total fragments that are intact, that is:

$$Z = \frac{x}{c}$$

The expected value of Z is P(no damage):

$$E [ Z ] = E [ P ( no damage ) ]$$

$$E [ \frac{x}{c} ] = E [ {( 1 - p )}^{n} ]$$

Focusing on the expectations, one may solve for p as:

$$p = 1 - \sqrt[n]{\frac{x}{c}}$$

In some embodiments two amplicons of different sizes are measured without measuring total nucleic acid content. One may assume that there are two amplicons of size n and m that are measured at concentration x₁ and x₂ respectively. Focusing on expected values, one may note that these variables follow a binomial distribution. In some embodiments one may assume that there are s total fragments of each in the original samples, some of which are intact and some of which are damaged. The total proportion of fragments detected for each is:

proportion fragment 1 intact=(1−p)ⁿ^{₂}

proportion fragment 2 intact=(1−p)ⁿ^{₂}

Assuming there was the same number of fragment 1 and 2 present initially, L, the ratio between the detected quantities of fragments is:

$$L = \frac{{x}_{1}}{{x}_{2}}$$

### Thus,

$$\frac{{( 1 - p )}^{{n}_{1}}}{{( 1 - p )}^{{n}_{2}}} = \frac{{x}_{1}}{{x}_{2}}$$

### Solving for p, we get:

$$p = 1 - \sqrt[{n}_{1} - {n}_{2}]{\frac{{x}_{1}}{{x}_{2 }}}$$

Based on these results, statements can be made about performance in other assays. For example, the portion of intact fragments on n nucleotides in length is:

(1−p)ⁿ (8)

Likewise, the distribution of errors in a fragment of length n can be calculated by as binom (n,p), with an average number of errors being expressed as:

np (9)

### DEFINITIONS

As used herein, “Geometric distribution” is given the standard meaning in the art.

As used herein, “quality” of a nucleic acid sample means the extent to which a nucleic acid sample is suitable or effective in a downstream application reliant upon nucleic acid integrity.

As used herein, “damage” means anything that reduces the quality of a nucleic acid sample. A non-limiting list of examples includes double stranded breaks, abasic sites, intrastrand cross links, inter strand cross links, nucleic acid-protein crosslinking, base modification and modification to the sugar backbone of a nucleic acid.

As used herein, “qPCR” means quantitative polymerase chain reaction. qPCR may involve a DNA template from a DNA sample or a DNA template that the product of a reverse-transcribed RNA sample.

As used herein, p is the proportion of damaged bases in a nucleic acid, k is the distance to the next damaging event on a nucleic acid strand, n and m are the length of an amplicon within a nucleic acid, L is the proportion of the detected intact nucleic acid to total nucleic acid, x is the amount of detected intact nucleic acid, c is the total amount of nucleic acid

As used herein, the ACq method means the method of calculating change in quantification cycle values in a quantitative PCR reaction.

As used herein, an “amplicon” is a nucleic acid fragment produced by the targeted amplification of a template via a PCR reaction involving paired oligonucleotide primers.

As used herein, a “manipulation” is any contacting of a substrate, such as a nucleic acid sample.

As used herein, a “nucleotide base position” refers to a base of a nucleic acid as well as its associated sugar (ribose or deoxyribose, for example) and adjacent phosphodiester backbone.

### EXAMPLES

### Example 1

RNA was extracted with the HighPure kit from Roche. RNA was quantified by UVspec to obtain a measurement, c, of total nucleic acids. Quantifast assays for the ACTB and 18S transcripts (Qiagen) were used to detect material present, and regression to a cell line (RNA from Agilent's Universal Cell line mixture) was used to fit concentrations for the samples. Using equation 1, the p-value of each sample was calculated for both 18S and ACTB.

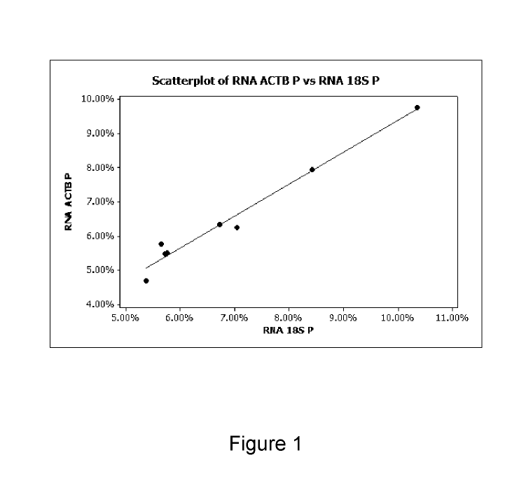

RNA is extracted from several tissues. 18S and ACTB amplifiable accumulation levels are assayed using qPCR. A value, p, is determined using the methods described herein. See FIG. 1. The graph indicates that the methods disclosed herein yielded values of p that were consistent across different regions interrogated by qPCR.

### Example 2

Primers and Probes suitable for the amplification of amplicons of distinct sizes for the comparison of amplification ratios. Primers and probes as shown below were used to amplify and to detect fragments of a locus in the 2p15 region and a HNRNPA1 transcript.

TABLE 1
RNA oligonucleotides (HNRNPA1 gene)

| | Forward Primer | Probe | Reverse Primer |
|---|---|---|---|
| 83 bp | GGGCTTTGCCTTTGTAA | TGACGACCATGACTCCGT | TGTGGCCATTCACAGTATGGT |
| Amplicon | CCTT | GGATA | A |
| | (SEQ ID NO: 1) | SEQ ID NO: 2) | (SEQ ID NO: 3) |
| 231 bp | GGACCCATGAAGGGAGG | TTTGGAGGCAGAAGCTCT | GCTTGGCTGAGTTCACAAATC |
| Amplicon | AAA | GGCC | (SEQ ID NO: 6) |
| | (SEQ ID NO: 4) | (SEQ ID NO: 5) | |

TABLE 2
DNA oligonucleotides (Chromosome 2p15)

| | Forward Primer | Probe | Reverse Primer |
|---|---|---|---|
| 87 bp | CAGCGTTGGTAGATCCT | TCTGGCCACACTTGAGTT | CTGCGAGTGCTGCGAGAAG |
| Amplicon | GACA | CCATGG | (SEQ ID NO: 9) |
| | (SEQ ID NO: 7) | (SEQ ID NO: 8) | |
| 250 bp | TTCCGACAGACCTTTCC | CCACCGTCTGTGGCCTGA | AGGTGAGGCGCGTAAAGGA |
| Amplicon | ACTC | GG | (SEQ ID NO: 12) |
| | (SEQ ID NO: 10) | (SEQ ID NO: 11) | |

The primers and probes were used under standard qPCR conditions with appropriate standards, and values were fit by regression. Ratios for amplification of the DNA and reverse-transcribed RNA templates for each amplicon were interpreted as the ratio of the large amplicon to the small amplicon where a larger ratio indicates more intact nucleic acid. Ratios were also interpreted using the disclosed equation 2 to determine a value of p for the DNA and RNA samples, respectively, and equation 3 was used to estimate the quantity of fragments c greater than a given length m.

Four standards were run and regression was used to determine the slope/intercept of the regression. The Ct values of the samples were fit to the regression equation to estimate the concentration of amplifiable template in the sample. For example, we assume that the primers in table 1 were used such that the 83 bp amplicon is amplicon 1 and the 231 bp amplicon is amplicon 2. If we obtained fitted values of 35 ng/μL for the template directing synthesis of amplicon 1 and 15 ng/μL for the template directing synthesis of amplicon 2, we would calculate the p-metric as follows:

$$p = 1 - \sqrt[{n}_{1} - {n}_{2}]{\frac{{x}_{1}}{{x}_{2}}}$$

$$p = 1 - \sqrt[33 - 231]{\frac{35}{15}}$$

$$p = 1 - 0.9943 = 0.0057 = 0.57 \%$$

Accordingly, in this Example, the proportion of damaged bases was 0.57%.

### Example 3

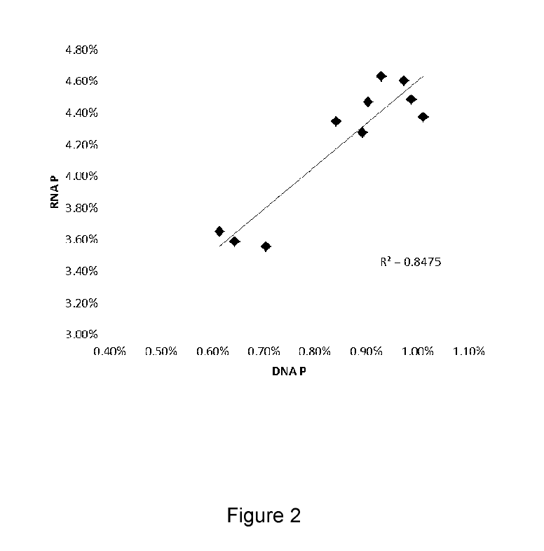

The value of p, the proportion of damaged bases, was measured on nucleic acids derived from 10 specimens using both DNA and RNA-derived templates. See FIG. 2. Extractions were conducted with the combined Allprep DNA/HighPure miRNA process previously discussed and were amplified via the primers in table 1 using either the Quantifast RT multiplex kit (Qiagen) for RNA or the Quantifast kit (Qiagen) for DNA according the manufacturer's instructions.

We observed a high level of correlation between the values for DNA and RNA. This was expected as formalin affects both types of nucleic acid in a similar way. This is strong evidence that we are measuring a real effect on the nucleic acids and not some artifact as the RNA and DNA sequences measured are completely unrelated to each other.

### Example 4

This Example relates to determining of the amount of nucleic acid from a given sample to be used in a downstream assay. PCR-based library creation for next-generation sequencing generally only works for fully intact fragments. A damaged fragment will not be amplified by PCR and therefore does not exist in the final product. In order to compensate for this, one can add more template if it is determined that nucleic acids from a given sample are damaged.

For example: if one wants to add 1 million amplifiable fragments of 400 bp long into a sequencing reaction. Assuming we knows that only 25% of them are undamaged, so that we can determine that adding 1 million fragments of sample-extracted nucleic acids will result in only 250,000 fragments of undamaged template. Using the methods disclosed herein, we can determine that we would need 1,000,000/0.25 fragments, or 4,000,000 fragments of the nucleic acids from the sample in question to get 1,000,000 amplifiable templates. Thus, we can therefore perform a downstream manipulation by adding 4 million fragments to our sequencing reaction so we end up with 1 million intact ones. This calculation to determine the proper number of starting molecules can be done, for example, with the effective quantity calculation for c as described herein.

### Example 5

Correction of Bias caused by FFPE stored samples. As a result of damage in the FFPE preservation and nucleic acid extraction processes, longer nucleic acid fragments obtained from these samples may be undercounted in assays that are sensitive to nucleic acid damage, such as qPCR or next-generation sequencing experiments.

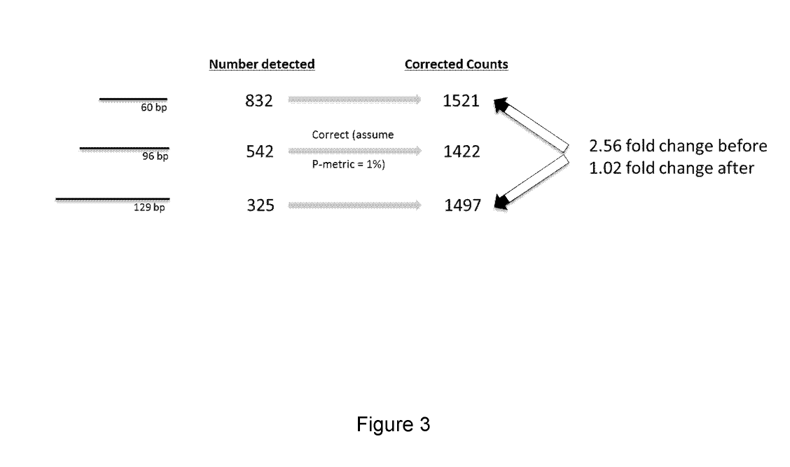

For example, FIG. 3 shows three fragments. They are different sizes, and in this Example we know that the larger fragments are more likely to be damaged than the small fragments. Thus, there is a bias against larger fragments and we know there are actually more than we originally detected.

The following equation may be used to correct for this damage:

y_{c}=yᵣ(1−p)^{m}

wherein y_{c}=the corrected quantification, yᵣ=the raw quantification given by the assay, p=the value of p, for example calculated by the methods disclosed herein, and m=the length of the region quantified. For example, if p=1%, m=200 and yᵣ=100 ng/μL, the value of y_{c} can be calculated as 13.4 ng/μL. This equation corrects for biases in the results due to nucleic acid damage. The equation is derived by dividing the raw read yᵣ by the probability that no damage occurs in a given fragment.

### Example 6

This Example shows the extraction results from 5 unique samples. Specimens were three months to one year old, were 0.3 cm to 1 cm on each dimension and were collected at multiple hospitals, each with its own varying methods. All specimens were fully embedded as FFPE samples and had not had previous slides cut.

Results are from 8×75 mm² 10 uM sections. Extractions were conducted with the combined Allprep DNA/HighPure miRNA process previously discussed.

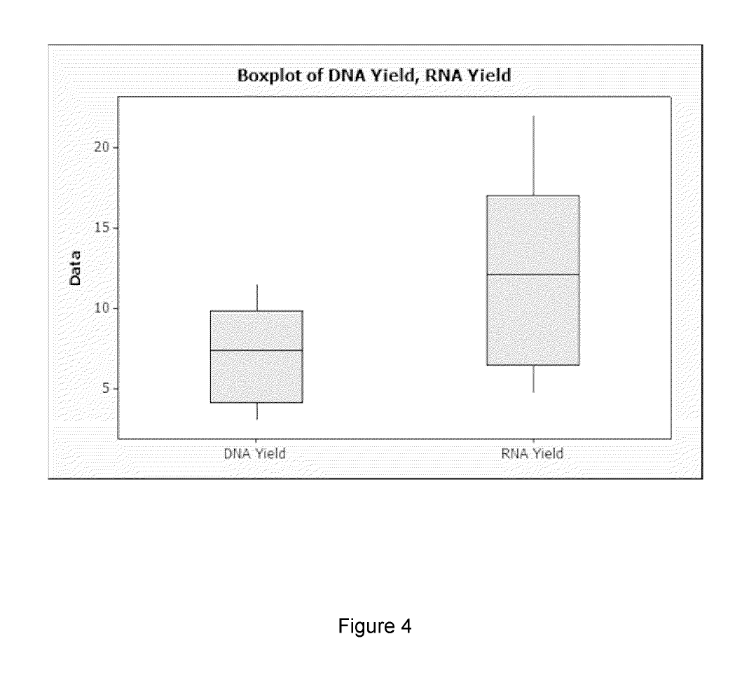

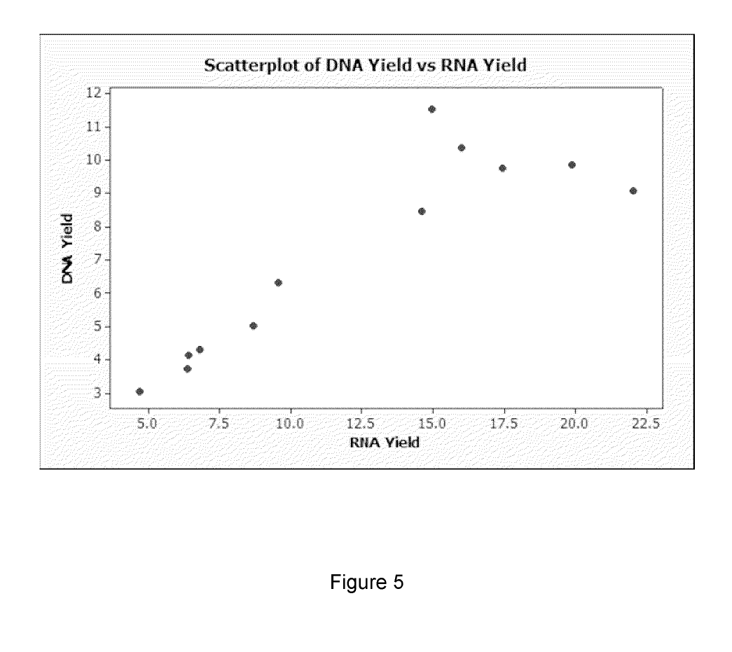

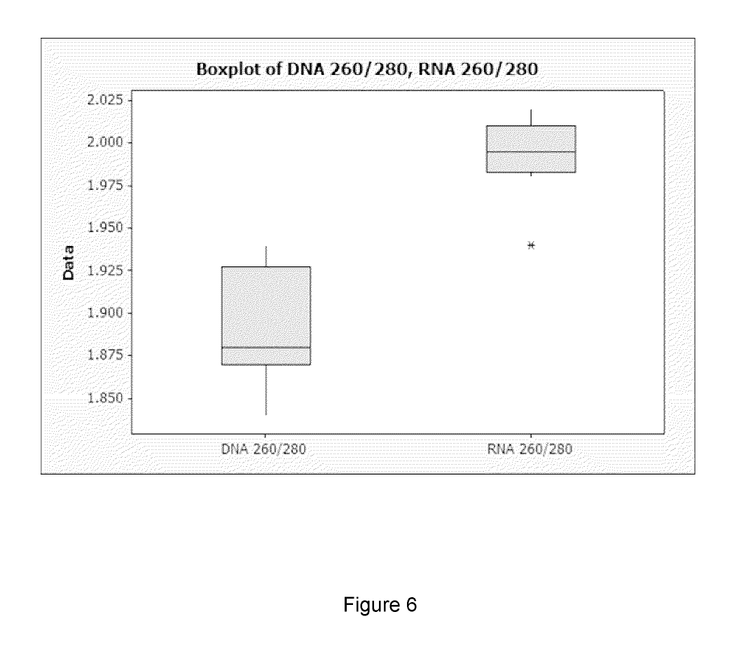

Results are indicated in FIGS. 4, 5, and 6.

### Example 7

This Example shows the use of a p metric to evaluate nucleic acid integrity. A sample is analyzed and a quality metric of p=0.92% is determined. It is concluded that 0.92% of bases in the sample (less than 1 in 100) were damaged. It is further concluded that 63% of 50 bp regions are intact, and that there are on average 0.46 errors in any 50 bp region in a sample.

Similarly, it is concluded that 40% of 100 bp regions are intact, and that on average there is a 0.92% chance of an error at any given base in a 100 bp region. It is concluded that 15% of 200 bp regions are intact, and that for the sample in question there is, again a 0.92% chance of an error at any given base in a 100 bp region.

### Example 8

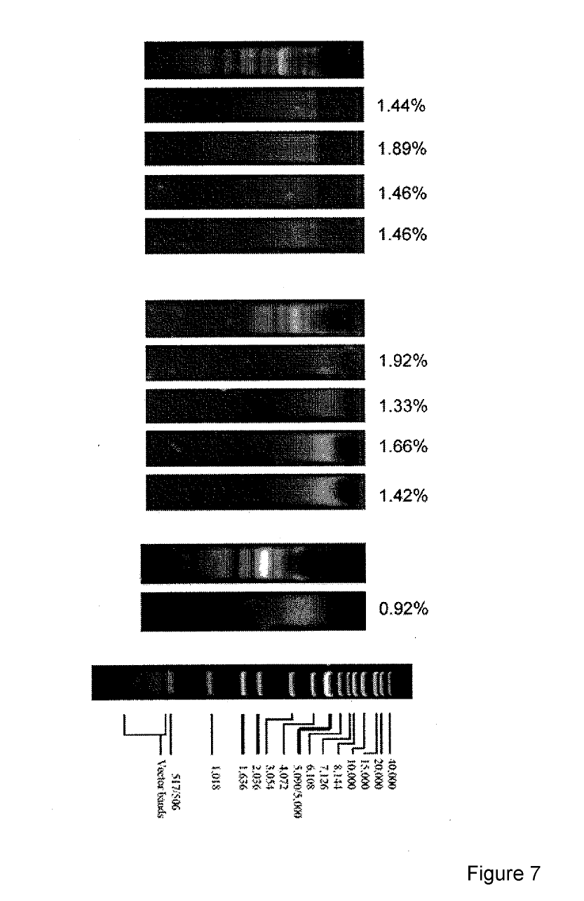

Comparison of p to DNA electrophoresis. FIG. 7 shows electrophoresis size fractionation of DNA with the measured p-metric listed on the right. The gel was run such that the DNA migrated from right to left. Fragments that are larger and experience fewer strand breaks tend to migrate farther towards the right. Observation of this image allows one to see that there is little correlation between the p-metric and the gel image. This indicates that the p-metric provides valuable information that cannot be obtained by traditional techniques.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

What is claimed is:

1. A method of performing manipulations on a sample of interest, comprising:
providing nucleic acids from a sample of interest;
measuring the amount of total nucleic acids provided from the sample;
amplifying a preselected region of the nucleic acids to form amplicons having a predetermined length such that only nucleic acids that do not contain damage are amplified;
measuring the amount of amplicons generated;
comparing said amount of amplicons obtained by amplification of said nucleic acids to a standard curve reflective of amounts of undamaged template that would yield similar amounts of template;
determining an amount of undamaged nucleic acids that would yield the measured amount of amplicons generated;
comparing the total nucleic acid amount to the determined amount of undamaged nucleic acids, wherein the comparison is indicative of a proportion of base positions in the nucleic acids from a sample of interest that are damaged; and
performing a further manipulation on said sample of interest if the proportion is below a threshold value.

2. The method of claim 1, wherein said comparing to a standard curve comprises comparing said amount of amplicons obtained by amplification of said nucleic acids to an amount of amplicons obtained by amplification of known quantities of undamaged total template nucleic acids.

3. The method of claim 1, wherein the proportion of base positions in the nucleic acids from a sample of interest is described as
$$p = 1 - \sqrt[n]{\frac{x}{c}} ,$$
and
c is the amount of total nucleic acid in said sample;
x is the equivalent amount of undamaged nucleic acids corresponding to the amount of amplifiable nucleic acids from a sample of interest of an amplicon of length n base positions within said sample; and
p is the proportion of base positions that are damaged in said sample.

4. The method of claim 1 wherein said further manipulation is a manipulation that is sensitive to cells having nucleic acid damage.

5. The method of claim 1 wherein said further manipulation comprises amplification of at least some of said nucleic acids from said sample of interest.

6. The method of claim 1 wherein said further manipulation comprises a reaction to determine a sequence of bases that comprise said nucleic acids from said sample of interest.

7. The method of claim 1 wherein said measuring the amount of total nucleic acid comprises spectrophotometric analysis.

8. The method of claim 1 wherein said measuring the amount of amplifiable nucleic acids from the amplicons comprises quantitative PCR.

9. The method of claim 1 wherein the sample of interest is a formalin fixed paraffin embedded (FFPE) tissue sample.

10. The method of claim 1 wherein said further manipulation comprises comparing said proportion to a threshold proportion value and discarding said nucleic acids if said proportion is above said threshold.

11. A method of determining the extent of damage to a tissue sample, comprising the steps of:
providing nucleic acids taken from a tissue sample;
measuring the amount of total nucleic acids in the tissue sample;
amplifying a preselected region of the nucleic acids to form amplicons having a predetermined length;
measuring the amount of amplicons generated;
comparing said amount of amplicons obtained by amplification of said nucleic acids to a standard curve reflective of amounts of undamaged template that would yield similar amounts of template;
determining an amount of undamaged nucleic acids that would yield the measured amount of amplicons generated;
comparing said total nucleic acid amount to said determined equivalent amount of undamaged nucleic acids, wherein the comparison is indicative of a proportion of base positions in the nucleic acids from a sample of interest that are damaged; and
determining the extent of damage to the tissue sample by comparing the assessed proportion to a threshold value.

12. The method of claim 11, wherein said comparing to a standard curve comprises comparing said amount of amplicons obtained by amplification of said nucleic acids to an amount of amplicons obtained by amplification of known quantities of undamaged total template nucleic acids.

13. The method of claim 11, wherein the proportion of base positions in the nucleic acids from a sample of interest is described as
$$p = 1 - \sqrt[n]{\frac{x}{c}} ,$$
and wherein
c is the amount of total nucleic acids in said sample;
x is the equivalent amount of undamaged nucleic acids corresponding to the amount of amplifiable nucleic acids from a sample of interest of an amplicon of length n base positions within said sample; and
p is the proportion of base positions that are damaged in said sample.

14. The method of claim 11, further comprising performing at least one additional manipulation of the tissue if the extent of damage is below a threshold value.

15. The method of claim 14, wherein said at least one further manipulation of said sample is sensitive to nucleic acid damage.

16. The method of claim 11, wherein said measuring the amount of total nucleic acids taken from a tissue sample comprises spectrophotometric analysis.

17. The method of claim 11, wherein said measuring the amount of amplified nucleic acid of an amplicon of length n base positions within said sample comprises quantitative PCR.

18. The method of claim 11, wherein said obtaining at least a nucleic acid sample comprises extracting said nucleic acid sample from a FFPE embedded sample.

19. A method of performing manipulations on a sample of interest, comprising:
providing nucleic acids from a sample of interest;
amplifying a preselected region of the nucleic acids to form a first amplicon of a predetermined length n₁;
amplifying a preselected region of the nucleic acids to form a second amplicon of a predetermined length n₂;
measuring the amount of amplified nucleic acids from of said first and second amplicon;
comparing the amount of the first and the second amplicon generated, wherein the comparison is indicative of a proportion of base positions in the template for the amplicons that are damaged; and
performing a further manipulation on said sample of interest.

20. The method of claim 19, wherein the proportion of base positions in the nucleic acids from a sample of interest is described as,
$$p = 1 - \sqrt[{n}_{1} - {n}_{2}]{\frac{{x}_{1}}{{x}_{2 }}} ,$$
wherein
c is the amount of total nucleic acid in said sample;
x₁ is the amount of amplified nucleic acid of said first amplicon of length n base positions within said sample; and
p is the proportion of base positions that are damaged in said sample.

21. The method of claim 19 wherein said further manipulation is a manipulation that is sensitive to nucleic acid damage.

22. The method of claim 19 wherein said further manipulation comprises amplification of at least some of said nucleic acid sample.

23. The method of claim 19 wherein said further manipulation comprises a reaction to determine a sequence of bases that comprise said nucleic acid sample.

24. The method of claim 19, wherein said measuring comprises quantitative PCR.

25. The method of claim 19 wherein the sample of interest is a formalin fixed paraffin embedded (FFPE) tissue sample.

26. The method of claim 19 wherein said further manipulation comprises comparing said proportion to a threshold proportion value and discarding said nucleic acids if said proportion is above said threshold.

27. A method of determining the extent of damage to a tissue sample, comprising the steps of:
providing nucleic acids taken from a tissue sample;
measuring the amount of total nucleic acids taken from the tissue sample;
amplifying a first preselected region of the nucleic acids to form amplicons having a first predetermined length n₁;
measuring the amount of amplifiable nucleic acids from the first amplicons;
amplifying a second preselected region of the nucleic acids to form a second amplicon of a predetermined length n₂;
measuring the amount of amplified nucleic acids from of said first and second amplicon;
comparing the amount of the first and the second amplicon generated, wherein the comparison is indicative of a proportion of base positions in the template for the amplicons that are damaged; and
determining the extent of damage to the tissue sample by comparing the assessed proportion to a threshold value.

28. The method of claim 27, wherein the proportion of base positions in the nucleic acids from a sample of interest is described as,
$$p = 1 - \sqrt[{n}_{1} - {n}_{2}]{\frac{{x}_{1}}{{x}_{2 }}} ,$$
wherein
x₁ is the amount of a first amplicon of length n₁ base positions within said sample; x₂ is the amount of a second amplicon of length n₂ base positions within said sample; and
p is the proportion of base positions that are damaged in said sample.

29. The method of claim 27, wherein said at least one further manipulation of said sample comprises amplification of at least some of said nucleic acid sample.

30. The method of claim 27, wherein said at least one further manipulation of said sample is sensitive to nucleic acid damage.

31. The method of claim 27, wherein said measuring comprises quantitative PCR.

32. The method of claim 27, wherein said obtaining at least a nucleic acid sample comprises extracting said nucleic acid sample from a FFPE embedded sample.

33. The method of claim 27, further comprising comparing said proportion to a threshold proportion value and discarding said nucleic acids if said proportion is above said threshold.
